(19) United States
(12) Patent Application Publication
(10) Pub. No.: US 2011/0077536 A1
(43) Pub. Date: Mar. 31, 2011

(54) BIOLOGICAL INFORMATION MANAGEMENT SYSTEM AND MEASUREMENT DEVICE

(75) Inventor: Nobuo KUBO, Kyoto-shi (JP)

(73) Assignee: OMRON HEALTHCARE CO., LTD., KYOTO-SHI (JP)

(21) Appl. No.: 12/964,308

(22) Filed: Dec. 9, 2010

Related U.S. Application Data
(63) Continuation of application No. PCT/JP2009/061632, filed on Jun. 25, 2009.

(30) Foreign Application Priority Data
Jul. 23, 2008 (JP) 2008-189589

(51) Int. Cl. A61B 5/00; G01G 19/50; A61B 5/022

(52) U.S. Cl. 600/499; 177/245; 600/300

(57) ABSTRACT

A biological information management system according to the present invention includes a measurement device for measuring biological information of a user and a management device for managing the biological information, wherein the measurement device includes measurement state information generation means for generating measurement state information representing a state in which the biological information is measured when the biological information is measured; and output means for outputting the biological information and the measurement state information generated when the biological information is measured, and wherein the management device includes reception means for receiving the measurement state information and the biological information which are outputted by the output means; and evaluation means for evaluating a reliability of the biological information received by the reception means based on the measurement state information received by the reception means.

### TECHNICAL FIELD

The present invention relates to a biological information management system for managing biological information of a user measured by a measurement device, and also relates to a measurement device.

### BACKGROUND ART

A biological information management system using a network is known as a technique for managing biological information such as a weight, a body composition, and a blood pressure.

The biological information management system includes a measurement device such as a weight scale, a body composition monitor, and a blood pressure monitor, and a management device for managing biological information measured by the measurement device. For example, the biological information management system is used for the purpose of health management of a user.

In the conventional biological information management system, however, the management device cannot determine whether the biological information is reliable or not.

Patent Document 1 discloses a measurement device for identifying a user based on a fingerprint. This measurement device can prevent “spoofing” of a user (i.e., another person performs measurement pretending as the user). However, even when this measurement device is used, a management device is unable to determine whether the user performs measurement according to a correct method.

Patent Document 2 discloses a blood pressure monitor for guiding a user to have a correct posture when the user does not have the correct posture. However, even when such a measurement device is used, biological information sent to a management device is not necessarily reliable (the user does not necessarily measure the biological information according to the guidance). In other words, the management device is unable to determine whether the user performs measurement according to a correct method. Moreover, some of users are unable to measure according to the correct method, and such users may feel uncomfortable when forced to measure according to the correct method (as a result, this may affect the measured biological information).

### PRIOR ART DOCUMENT

### Patent Documents

Patent Document 1: Japanese Unexamined Patent Publication No. 2003-299625
Patent Document 2: Japanese Unexamined Patent Publication No. 2003-102693

### SUMMARY OF THE INVENTION

### Problem to be Solved by the Invention

The present invention has been made in view of the above circumstances, and an object of the present invention is to provide a biological information management system that uses a simple method to determine whether biological information sent to a management device is reliable or not, and also to provide a management device.

### Means for Solving the Problem

In order to achieve the above object, the present invention employs the following structure.

A biological information management system according to the present invention includes a measurement device for measuring biological information of a user and a management device for managing the biological information, wherein the measurement device includes measurement state information generation means for generating measurement state information representing a state in which the biological information is measured when the biological information is measured; and output means for outputting the biological information and the measurement state information generated when the biological information is measured, and wherein the management device includes reception means for receiving the measurement state information and the biological information which are outputted by the output means; and evaluation means for evaluating a reliability of the biological information received by the reception means based on the measurement state information received by the reception means.

According to this structure, the measurement device generates and outputs the biological information as well as the measurement state information representing the state in which the biological information is measured. Then, the management device evaluates the reliability of the biological information based on the measurement state information. In other words, whether the biological information is reliable or not can be determined by the simple method using the measurement state information.

The measurement device preferably has a function of measuring a blood pressure, and when the blood pressure is measured as the biological information, the measurement state information is preferably information representing at least any one of an angle of a cuff unit arranged on the measurement device for measuring the blood pressure, a variation of an acceleration of the cuff unit, and a variation of a pressure in the cuff unit during measurement. With this structure, whether the blood pressure value sent to the management device is reliable or not can be determined.

The measurement device preferably has a function of measuring a weight, and when the weight is measured as the biological information, the measurement state information is preferably information representing a load value used for a zero-point calibration of the weight of the measurement device or a variation of a load during measurement. With this structure, whether the weight value sent to the management device is reliable or not can be determined.

The measurement device preferably has a function of measuring a body composition, and when the body composition is measured as the biological information, the measurement state information is preferably information representing a variation of the body composition or a variation of impedance during measurement. With this structure, whether the body composition value sent to the management device is reliable or not can be determined.

The measurement device preferably has a function of measuring a weight and a body composition, and when the weight is measured as the biological information, the measurement state information is preferably information representing a variation of the body composition or a variation of impedance during measurement. With this structure, whether the weight value sent to the management device is reliable or not can be determined.

The measurement state information is preferably a time it takes for the user to measure the biological information with the measurement device. When it takes a long time to measure the biological information, the user is likely to be unaccustomed to measurement, or the user is likely to try to do cheating. With this structure, whether the biological information sent to the management device is reliable or not can be determined.

The management device preferably includes storage means for storing advices about measurement methods in association with the measurement state information and advice output means for outputting an advice corresponding to the measurement state information received by the reception means from among the advices stored in the storage means. When the management device transmits an advice to a user according to a measurement state (information), the administrator of the management device needs to check the measurement state information, and transmit an advice. With this structure, advices are automatically outputted according to measurement state, and therefore, it is less cumbersome for the administrator. Further, the user is caused to understand that, when the user performs measurement according to a cheating measurement method, the user's measurement according to the cheating measurement method is known (to the administrator) and that the measurement method is a cheating. Therefore, cheating measurement can be reduced. On the other hand, the user is caused to understand that, when the user performs measurement according to a correct measurement method, the measurement method is correct. Therefore, the satisfaction of the user is considered to improve.

The management device is preferably able to change a criterion of evaluation performed by the evaluation means. The criteria for the evaluation performed by the evaluation means may be different for each person (for example, in many cases, a posture of a young person and a posture of an elderly person are different, and it is not appropriate to use the same criteria to make determination on such persons). With this structure, criteria appropriate for each person can be set, and whether the biological information is reliable or not can be determined more accurately.

The management device preferably includes display means causing a display unit of the management device to display the biological information in a pattern according to the evaluation result of the evaluation means. For example, the display means preferably causes the display unit of the management device to display a graph of the biological information with a dot color, a dot shape, and/or a line type, according to the evaluation result of the evaluation means. With this structure, the administrator of the management device can easily determine whether biological information is reliable or not by just looking at the biological information displayed on the display unit.

The display means preferably causes the display unit of the management device to display only biological information in which the evaluation result of the evaluation means is a predetermined evaluation result. With this structure, only reliable biological information or only unreliable biological information can be displayed on the display unit. For example, by displaying only the reliable biological information, the administrator of the management device can accurately analyze the biological information of the user.

A measurement device according to the present invention measures biological information of a user, and the measurement device includes measurement state information generation means for generating measurement state information representing a state in which the biological information is measured when the biological information is measured, and output means for outputting the biological information and the measurement state information generated when the biological information is measured, wherein the measurement state information is used by a management device managing the biological information to evaluate reliability of the biological information.

With this structure, the measurement device generates the biological information as well as the measurement state information representing the state in which the biological information is measured. Then, the generated measurement state information is used by the management device to evaluate the reliability of the biological information. In other words, whether the biological information is reliable or not can be determined by the simple method using the measurement state information.

### EFFECT OF THE INVENTION

According to the present invention, the biological information management system is provided that uses the simple method to determine whether biological information sent to a management device is reliable or not, and the management device is also provided.

### BRIEF DESCRIPTION OF THE DRAWINGS

FIG. 1 is a block diagram illustrating an example of structure of a biological information management system according to the present embodiment.

FIG. 2 is a block diagram illustrating an example of structure of a measurement device according to the present embodiment.

FIG. 3 is a block diagram illustrating an example of structure of an administrator's terminal according to the present embodiment.

FIG. 4 is a graph illustrating an example of display of biological information.

FIG. 5 is a graph illustrating an example of display of biological information.

FIG. 6 is a diagram illustrating a determination method performed by an evaluation unit and measurement state information of a measurement device according to a first embodiment.

FIG. 7 is a diagram illustrating a determination method performed by the evaluation unit and measurement state information of the measurement device according to the first embodiment.

FIG. 8 is a diagram illustrating an example of output data from the measurement device according to the first embodiment.

FIG. 9 is a graph illustrating a determination method performed by an evaluation unit and measurement state information of a measurement device according to a second embodiment.

FIG. 10 is a graph illustrating a determination method performed by the evaluation unit and measurement state information of the measurement device according to the second embodiment.

FIG. 11 is a diagram illustrating an example of output data from the measurement device according to the second embodiment.

FIG. 12 is a flowchart illustrating an example of flow of measurement performed by a measurement device according to a fourth embodiment.

FIG. 13 is a diagram illustrating an example of output data from the measurement device according to the fourth embodiment.

FIG. 14 is a graph illustrating a determination method performed by an evaluation unit and measurement state information of a measurement device according to a fifth embodiment.

FIG. 15 is a graph illustrating the determination method performed by the evaluation unit and measurement state information of the measurement device according to the fifth embodiment.

FIG. 16 is a diagram illustrating an example of output data from the measurement device according to the fifth embodiment.

FIG. 17 is a graph illustrating a determination method performed by an evaluation unit and measurement state information of a measurement device according to sixth and seventh embodiments.

FIG. 18 is a graph illustrating the determination method performed by the evaluation unit and measurement state information of the measurement device according to the sixth and seventh embodiments.

FIG. 19 is a diagram illustrating an example of output data from the measurement device according to the sixth embodiment.

FIG. 20 is a graph illustrating the determination method performed by the evaluation unit and measurement state information of the measurement device according to the seventh embodiment.

FIG. 21 is a diagram illustrating an example of output data from the measurement device according to the seventh embodiment.

FIG. 22 is a block diagram illustrating an example of structure of an administrators terminal according to a modification.

### BEST MODE FOR CARRYING OUT THE INVENTION

In the past, a biological information management system has been used for the purpose of health management of users. For example, the system can be used by a doctor to understand health condition of a patient and give appropriate advice to the patient as necessary, or the system can be used by an employee of a health insurance company to understand health condition of an insured person (in order to assess premium). Accordingly, an administrator of the system is considered to be a doctor, an employee of a health insurance company, and the like.

With this biological information management system, a service for adding monetary value to biological information of a user can be achieved. For example, a service for giving a certain reward to a person who can sufficiently perform health management can be achieved (more specifically, a service for giving money points to a user according to a reduced weight when the user reduces his or her weight so as to get closer to an ideal weight).

Therefore, the biological information used by the biological information management system has to be reliable. Compared with conventional biological information management systems, the biological information management system according to the present embodiment uses measurement state information representing a state in which biological information is measured, thereby capable of determining whether the biological information used by the biological information management system is reliable or not. Hereinafter, the biological information management system according to the present embodiment will be described in detail with reference to the drawings.

### <System Configuration>

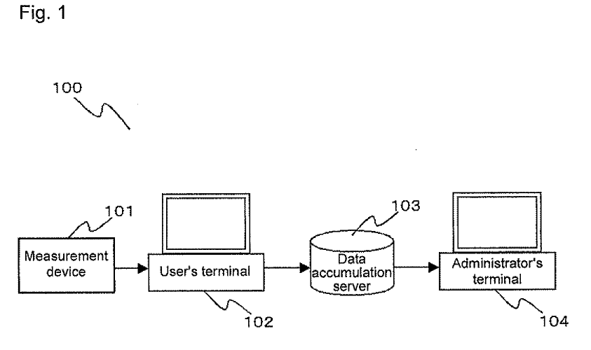

First, an example of constituting of the biological information management system according to the present embodiment will be described. FIG. 1 is a block diagram illustrating the example of structure of the biological information management system according to the present embodiment. As shown in FIG. 1, the biological information management system 100 according to the present embodiment includes a measurement device 101, a user's terminal 102, a data accumulation server 103, an administrator's terminal (management device) 104, and the like.

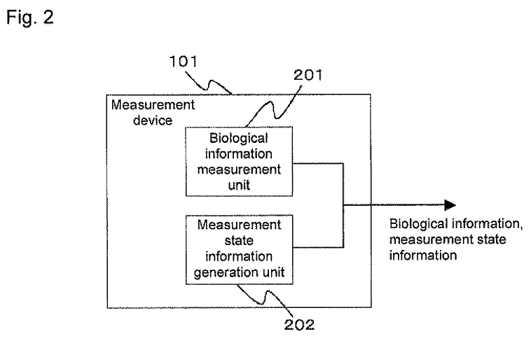

The measurement device 101 measures biological information of a user. The measurement device 101 uses a blood pressure monitor, a weight scale, a body composition monitor, and the like. FIG. 2 is a block diagram illustrating an example of structure of the measurement device 101 according to the present embodiment. As shown in FIG. 2, the measurement device 101 according to the present embodiment includes a biological information measurement unit 201, a measurement state information generation unit 202, and the like.

The biological information measurement unit 201 is a function for measuring the biological information of the user.

The measurement state information generation unit 202 is a function for generating measurement state information when the biological information is measured. The measurement state information represents a state in which the biological information is measured. For example, the measurement state information serves as an index for determining whether the user measures the biological information according to a correct method or not. When the measurement method is correct, the biological information is likely to be reliable. When the measurement method is incorrect, the biological information is likely to be unreliable. Accordingly, the reliability of the biological information can be evaluated from the above measurement state information. Specific examples of measurement state information will be described in detail later. When a blood pressure is measured as biological information, examples of measurement state information include information representing, during measurement, an angle of a cuff unit for measuring a blood pressure arranged in the measurement device, a variation of acceleration of the cuff unit, a variation of pressure in the cuff unit, and the like. Regarding “during measurement”, it may be any period as long as information for determining whether the measurement method is correct or not can be obtained. This measurement period varies according to measurement state information, and therefore, the details will be described later when the specific examples of measurement state information are described.

The measurement device 101 according to the present embodiment outputs, to the outside, the biological information and the measurement state information generated when the biological information is measured.

The user's terminal 102 is a terminal used by the user to transmit the biological information and the measurement state information to an administrator (administrator's terminal 104). The user's terminal 102 may be a personal computer (PC), a portable telephone, and the like. It should be noted that the function of the user's terminal 102 may be arranged on the measurement device 101. In the present embodiment, the user's terminal 102 is a PC, and the user's terminal 102 uploads the biological information and the measurement state information to the later-described data accumulation server 103 via a LAN. Data may be transmitted and received between the measurement device 101 and the user's terminal 102 via wires such as a LAN cable, a USB cable, and the like, or wirelessly by Bluetooth and the like.

The data accumulation server 103 is a server for storing and accumulating the biological information and the measurement state information which are outputted by the user's terminal 102. The measurement state information is accumulated in the data accumulation server 103 in such a manner that the measurement state information can be viewed by the user and the administrator. The function of the data accumulation server 103 may be arranged in the later-described administrator's terminal 104.

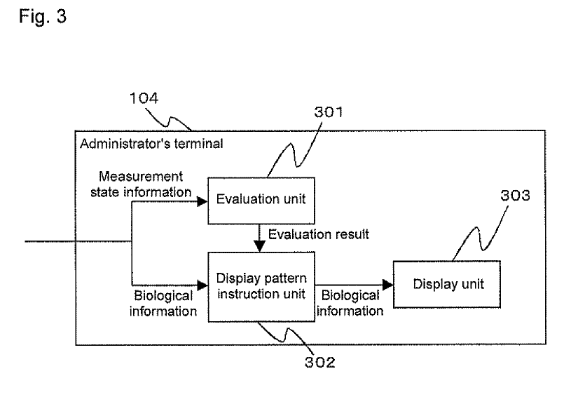

The administrator's terminal 104 obtains (receives) the biological information and the measurement state information outputted from the measurement device 101, and manages the biological information of the user. The administrator's terminal 104 may be a personal computer (PC), a portable telephone, and the like. In the present embodiment, the administrator's terminal 104 is a PC and the administrator's terminal 104 obtains (receives) the biological information and the measurement state information from the data accumulation server 103 via a LAN. FIG. 3 is a block diagram illustrating an example of structure of the administrator's terminal 104 according to the present embodiment. As shown in FIG. 3, the administrator's terminal 104 according to the present embodiment includes an evaluation unit 301, a display pattern instruction unit 302, a display unit 303, and the like.

The evaluation unit 301 evaluates the reliability of the biological information obtained together with the measurement state information, based on the obtained measurement state information. In the present embodiment, the biological information is evaluated into two types, i.e., reliable biological information or unreliable biological information. Specific examples of processings performed by the evaluation unit 301 (evaluation processings) will be described in detail later. In the biological information management system according to the present embodiment, the management device evaluates the measurement state information. Therefore, the user is less likely to know the criteria of determination.

The display pattern instruction unit 302 causes the display unit 303 of the administrator's terminal 104 to display biological information in a pattern according to the evaluation result of the evaluation unit 301. The display unit 303 may be, for example, a display device such as a liquid crystal display.

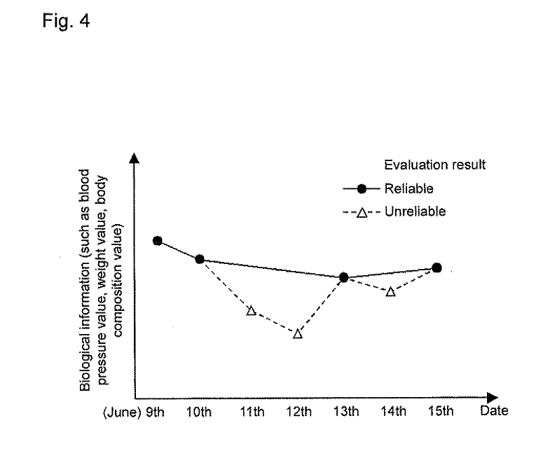

In the present embodiment, the display unit 303 displays a graph of biological information (for example, a graph in which a horizontal axis represents a date and a vertical axis represents biological information such as a blood pressure, a weight, a body composition) as shown in FIG. 4. In this case, the pattern according to the evaluation result of the evaluation unit 301 preferably uses dot colors, dot shapes, and line types. Biological information determined by the evaluation unit 301 to be reliable and biological information determined to be unreliable are represented using any one of different dot colors, different dot shapes, and different line types or using all of different dot colors, different dot shapes, and different line types. In this case, for example, the line type means a line type of a line connecting between pieces of biological information adjacent to each other in terms of time, and the line type of the line connecting the unreliable biological information may be different from the line types of other lines. Accordingly, the administrator can instantly, easily determine whether biological information is reliable or not. In the graph, when biological information between two pieces of reliable biological information is unreliable biological information, the two pieces of biological information may be connected via a line (in a case where line types are used as patterns, the two pieces of biological information are preferably connected by a line of the same line type as the line type of biological information determined to be reliable). Accordingly, the administrator can easily understand how the reliable biological information has changed.

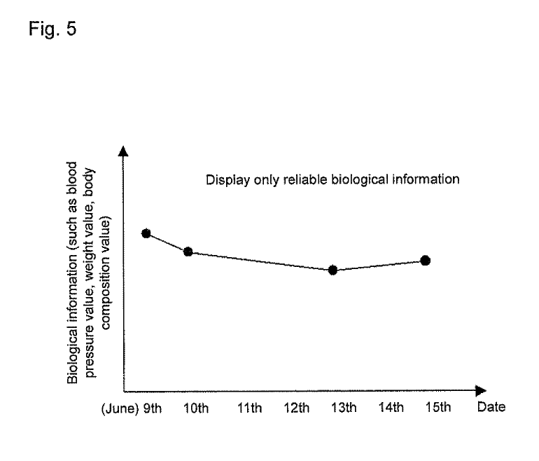

It should be noted that the display pattern instruction unit 302 may cause the display unit 303 to display only biological information in which the evaluation result of the evaluation unit 301 is a predetermined evaluation result. For example, the display pattern instruction unit 302 may cause the display unit 303 to display only reliable biological information. Accordingly, as shown in FIG. 5, the administrator can check only the reliable biological information, and therefore, the administrator can intuitively, correctly analyze the biological information. Further, the display pattern instruction unit 302 may cause the display unit 303 to display only unreliable biological information. Accordingly, the administrator can check only the unreliable biological information. When such information is obtained, the administrator is considered to obtain further knowledge about the measurement method of the user (for example, how frequently the user performs cheating measurement).

It should be noted that the display unit 303 may display only the current biological information of the user instead of the graph. In such a case, different colors may be used to display biological information according to evaluation results, and the evaluation result as well as the reliability (reliable, unreliable, and the like) may be displayed. A circle mark may be attached to reliable biological information, and an X mark may be attached to unreliable biological information.

As described above, according to the biological information management system according to the present embodiment, the measurement device generates and outputs the biological information as well as the measurement state information. On the other hand, the management device evaluates the reliability of the biological information based on the measurement state information. In other words, whether the biological information is reliable or not can be determined by the simple method using the measurement state information.

Further, in the present embodiment, the management device is configured to have the display pattern instruction unit. Accordingly, the administrator can know the health condition of the user with only the reliable biological information. Therefore, the administrator can give appropriate advice about the health to the user.

It should be noted that upload of information from the user's terminal 102 to the data accumulation server 103 and download of information from the administrator's terminal 104 in the data accumulation server 103 may not be performed via a LAN. For example, information may be transmitted and received via a wide area network such as the Internet.

Specific examples of measurement state information and evaluation processing will be hereinafter described in detail (first to seventh embodiments).

### First Embodiment

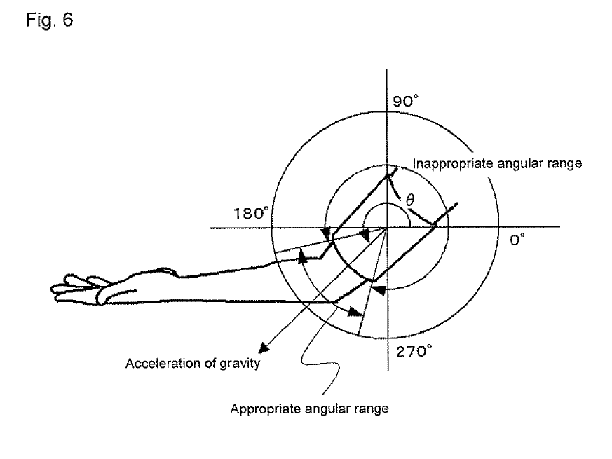

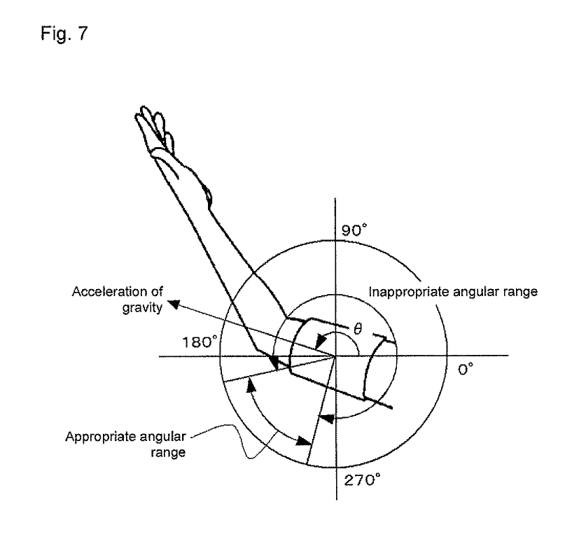

In the description of the first embodiment, the measurement device has a function of measuring a blood pressure, and a blood pressure is measured as biological information. As shown in FIG. 6, in order to correctly measure the blood pressure, an angle (for example, an angle represented by θ in the figure) of a cuff unit (band wrapped around a finger, a wrist, an arm, and the like to measure the blood pressure) is desired to be appropriate during measurement. In a case where the angle of the cuff unit (cuff angle) is not appropriate during measurement, the user is likely to be making a mistake in the measuring method or is likely to try to do cheating. The blood pressure value measured in such a condition is unreliable (for example, as shown in FIG. 7, the blood pressure value is calculated lower than it actually is when the arm is raised upward). Accordingly, in the first embodiment, information representing the angle of the cuff unit during measurement is used as measurement state information. Whether the measurement method is correct or not can be determined if the angle of the cuff unit can be obtained, for example, at a start of pressurizing process, from the start of pressurizing process to the end of the measurement, and at the end of the measurement. Therefore, these are referred to as “during measurement” in the present embodiment. It should be noted that periods other than the above periods may be adopted as “during measurement” as long as whether the measurement method is correct or not can be determined. In the description of the present embodiment, the cuff unit is assumed to be wrapped around an arm of the user.

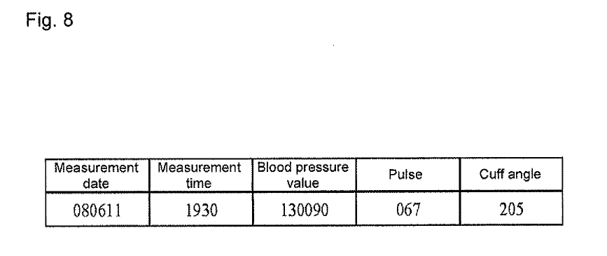

In the present embodiment, an angle sensor is arranged on the cuff unit. The angle sensor may be any device as long as it can measure an angle such as an acceleration sensor (FIGS. 6, 7 are examples where an acceleration sensor is used as the angle sensor). As shown in FIG. 8, the measurement device 101 outputs data (output data) including a measurement date, a measurement time, a blood pressure value, a pulse, a cuff angle, and the like (FIG. 8 is an example of output data including a blood pressure of 130/90 mmHg, a pulse of 67 beats, a cuff angle of 205 degrees, measured at 19:30 on Jun. 11, 2008). It should be noted that the cuff angle may be defined in anyway. The value of the cuff angle is determined according to the definition.

Then, the evaluation unit 301 determines whether the biological information is reliable or not according to the cuff angle, and the display pattern instruction unit 302 causes the display unit 303 to display the biological information in a pattern according to the evaluation result of the evaluation unit 301. In the present embodiment, the measurement condition is correct when the cuff angle is in a range of 190 to 260 degrees. Accordingly, in this condition, the evaluation unit 301 evaluates the biological information as “reliable”. When the cuff angle is out of the range, the measurement method is incorrect, and accordingly, the evaluation unit 301 evaluates the biological information as “unreliable”. Therefore, the biological information measured in the state shown in FIG. 6 can be evaluated as “reliable”, and the biological information measured in the state shown in FIG. 7 can be evaluated as “unreliable”.

### Second Embodiment

In the description of a second embodiment, the measurement device has a function of measuring a blood pressure, and a blood pressure is measured as biological information. In order to correctly measure the blood pressure, the user desirably remains stationary during measurement. In the second embodiment, a determination is made as to whether biological information is reliable or not according to whether the user remains stationary during measurement. More specifically, information representing a variation of acceleration of the cuff unit during measurement is used as measurement state information to determine whether the user remains stationary during measurement. Whether the measurement method is correct or not can be determined by finding the acceleration of the cuff unit in a period including a period of blood pressure measurement (for example, a period from the start of pressurizing process to the end of pressurizing process. Therefore, such a period is referred to as “during measurement” in the present embodiment. It should be noted that periods other than the above periods may be adopted as “during measurement” as long as whether the measurement method is correct or not can be determined.

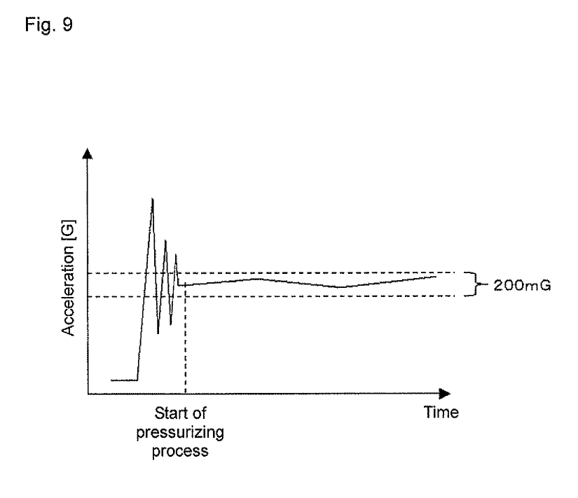

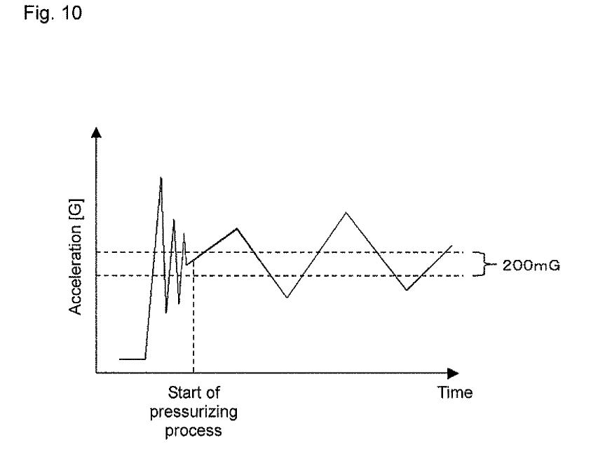

In general, when the user remains stationary during blood pressure measurement, the movement (shaking) of the cuff unit is small. When the user is moving, the shaking of the cuff unit is large. Therefore, whether the user remains stationary or not during measurement can be determined from the variation of acceleration of the cuff unit during measurement. Accordingly, in the present embodiment, an acceleration sensor is arranged on the cuff unit. The evaluation unit 301 evaluates whether the biological information is reliable or not according to the variation of acceleration of the cuff unit during measurement. A specific example of evaluation method will be described with reference to FIGS. 9 and 10.

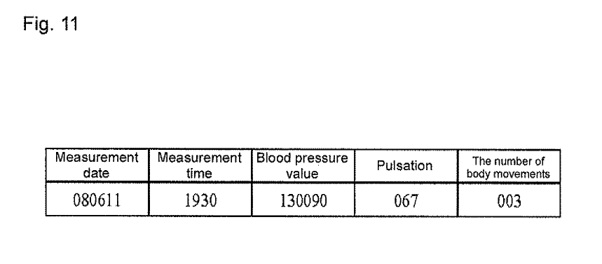

In the present embodiment, when the amplitude of output waveform of the acceleration sensor is less than 200 mG, the user is considered to remain stationary (FIG. 9), and when an amplitude of output waveform of the acceleration sensor is equal to or more than 200 mG, the user is considered to be moving (FIG. 10). More specifically, the measurement device 101 counts, as the number of body movements, the number of portions where the amplitude is equal to or more than 200 mG in the output waveform of the acceleration sensor during measurement. As shown in FIG. 11, the measurement device 101 outputs data (output data) including a measurement date, a measurement time, a blood pressure value, a pulse, the number of body movements, and the like (FIG. 11 is an example of output data including a blood pressure of 130/90 mmHg, a pulse of 67 beats, the number of body movements of 3 times, measured at 19:30 on Jun. 11, 2008).

Then, the evaluation unit 301 determines whether the biological information is reliable or not according to the number of body movements, and the display pattern instruction unit 302 causes the display unit 303 to display the biological information in a pattern according to the evaluation result of the evaluation unit 301. More specifically, the evaluation unit 301 evaluates the biological information as “reliable” when the number of body movements is less than a predetermined number, and evaluates the biological information as “unreliable” when the number of body movements is equal to or more than the predetermined number.

In the above description, the measurement device 101 counts and outputs the number of body movements. Alternatively, the measurement device 101 may output an output waveform (all output values) of the acceleration sensor during measurement, and the administrator's terminal 104 may analyze the output waveform. Still alternatively, instead of the number of body movements, the biological information may be evaluated according to the maximum acceleration detected by the acceleration sensor during measurement (for example, when the maximum acceleration is determined to be equal to or more than a predetermined threshold value, the biological information is evaluated as “unreliable”), or the maximum acceleration and the number of body movements may be used in combination.

### Third Embodiment

In the description of a third embodiment, the measurement device has a function of measuring a blood pressure, and a blood pressure is measured as biological information. The third embodiment is the same as the second embodiment in that a determination is made as to whether biological information is reliable or not according to whether the user remains stationary during measurement. More specifically, information representing a variation of pressure in the cuff unit during measurement is used as measurement state information to determine whether the user remains stationary during measurement. It should be noted that the periods defined in the same manner as the second embodiment may be adopted as “during measurement”.

In general, when the user remains stationary during blood pressure measurement, a fluctuation of the pressure in the cuff unit is small. When the user is moving, the fluctuation of the pressure in the cuff unit is large. Therefore, whether the user remains stationary or not during measurement can be determined from the variation of the pressure in the cuff unit during measurement. Accordingly, in the present embodiment, a pressure sensor is arranged on the cuff unit to measure the pressure in the cuff unit. The evaluation unit 301 evaluates whether the biological information is reliable or not according to the variation of the pressure in the cuff unit during measurement.

The specific evaluation method is the same as that of the second embodiment. For example, when an amplitude of output waveform of the pressure sensor is less than a predetermined threshold value, the user is considered to be stationary, and when an amplitude of output waveform of the pressure sensor is equal to or more than the predetermined threshold value, the user is considered to be moving. More specifically, the measurement device 101 counts, as the number of body movements, the number of portions where the amplitude is equal to or more than the threshold value in the output waveform of the pressure sensor during measurement. The measurement device 101 outputs data (output data) including a measurement date, a measurement time, a blood pressure value, a pulse, the number of body movements, and the like.

Then, the evaluation unit 301 evaluates whether the biological information is reliable or not according to the number of body movements, and the display pattern instruction unit 302 causes the display unit 303 to display the biological information in a pattern according to the evaluation result of the evaluation unit 301. More specifically, the evaluation unit 301 evaluates the biological information as “reliable” when the number of body movements is less than a predetermined number, and evaluates the biological information as “unreliable” when the number of body movements is equal to or more than the predetermined number.

In the above description, the measurement device 101 counts and outputs the number of body movements. Alternatively, the measurement device 101 may output an output waveform (all output values) of the pressure sensor during measurement, and the administrator's terminal 104 may analyze the output waveform. Still alternatively, instead of the number of body movements, the biological information may be evaluated according to the maximum pressure detected by the pressure sensor during measurement (for example, when the maximum pressure is determined to be equal to or more than a predetermined threshold value (it is to be understood that this threshold value is different from the above-mentioned threshold value), the biological information is evaluated as “unreliable”), or the maximum pressure and the number of body movements may be used in combination.

### Fourth Embodiment

In the description of the fourth embodiment, the measurement device has a function of measuring a weight, and a weight is measured as biological information (in other words, a weight scale is used as the measurement device).

In general, a weight scale performs a zero-point calibration of load when the weight scale is turned on. More specifically, when the weight scale is turned on, the load exerted on the weight scale is set as 0 kg. Therefore, when the user intentionally applies load during the zero-point calibration, the zero-point becomes wrong, whereby the user can cause the measurement device to calculate a false weight value. Accordingly, in the present embodiment, in order to evaluate whether the weight value is such a false weight value or not, information representing a load value used for the zero-point calibration during measurement is used as measurement state information. In the present embodiment, a time when the zero-point calibration of load is carried out (for example, when the power is turned on) is referred to as “during measurement”.

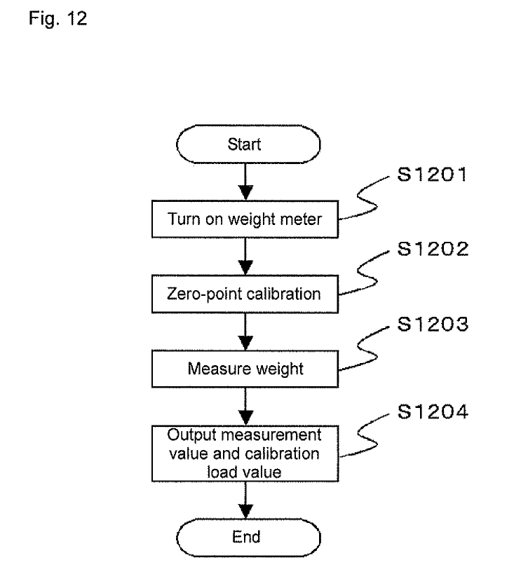

A flow of measurement performed by the measurement device (weight scale) according to the present embodiment will be described with reference to a flowchart of FIG. 12.

First, when the user turns on the weight scale (step S1201), the weight scale performs the zero-point calibration (step S1202). More specifically, the weight scale stores, as a load value (calibration load value) for zero-point calibration, a value of load exerted on the weight scale when the weight scale is turned on, and causes a display unit of the weight scale to display a value (0 kg) obtained by subtracting the thus stored load value. For example, the calibration load value is stored to a storage medium such as a non-volatile memory arranged in the weight scale.

Then, the user steps on the weight scale, and the weight is measured (step S1203). At this occasion, the display unit of the weight scale displays, as the weight of the user, a value obtained by subtracting the calibration load value from the actual load detected by the weight scale. When the weight (load) is stabilized, the weight is determined (calculated), and the measurement is finished.

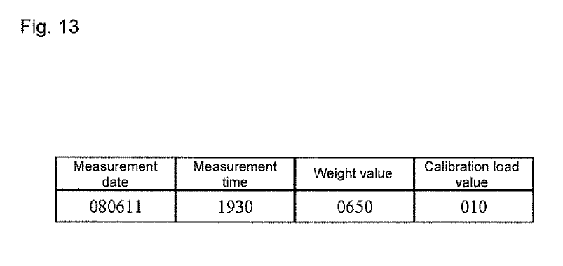

When the measurement is finished, the measurement value (weight value; biological information) and the calibration load value are outputted (step S1204). More specifically, as shown in FIG. 13, the measurement device 101 outputs data (output data) including a measurement date, a measurement time, a weight value, a calibration load value, and the like (FIG. 13 is an example of output data including a weight of 65.0 kg, a calibration load value of 1 kg, measured at 19:30 on Jun. 11, 2008). Then, the evaluation unit 301 evaluates whether the biological information is reliable or not according to the calibration load value (for example, when the calibration load value is within a predetermined range (such as ±2 kg), the biological information is evaluated as “reliable”, and when the calibration load value is out of the range, the biological information is evaluated as “unreliable”), and the display pattern instruction unit 302 causes the display unit 303 to display the biological information in a pattern according to the evaluation result of the evaluation unit 301.

### Fifth Embodiment

In the description of the fifth embodiment, the measurement device has a function of measuring a weight, and a weight is measured as biological information (in other words, a weight scale is used as the measurement device).

In general, a weight scale calculates, as a weight value, an average value and the like when a load value becomes stable. Therefore, there is a possibility of cheating, i.e., the user places, instead of the user, an object (solid material) having about the same weight as a human on the weight scale so as to cause the weight scale to calculate a false weight value.

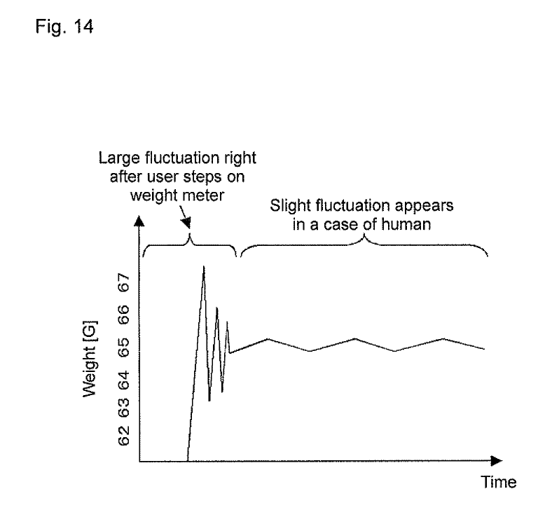

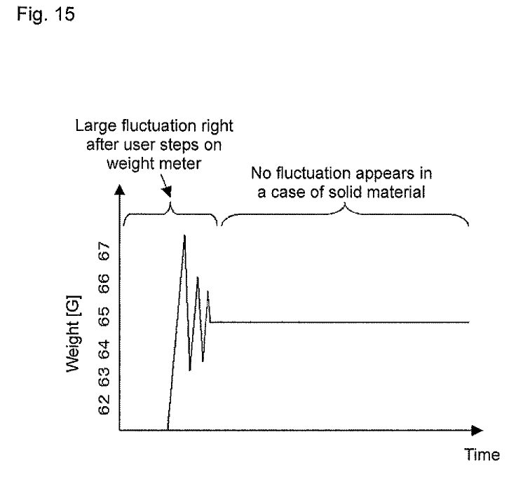

In general, when a human is on the weight scale, an output value (load value) of the weight scale during measurement fluctuates little as shown in FIG. 14. On the other hand, when the solid material is on the weight scale, the load value during measurement is substantially constant as shown in FIG. 15. Therefore, whether a human or a solid material is on the weight scale can be determined from the variation of load value during measurement. Accordingly, in the present embodiment, in order to evaluate whether a human is on the weight scale or not, information representing the variation of load value during measurement is used as measurement state information. Whether a human is on the weight scale or not can be determined if the variation of load value can be obtained in a predetermined period from when a measurement target (user) steps on the weight scale to when the weight value is calculated. Therefore, such a period is referred to as “during measurement” in the present embodiment. However, since the fluctuation (variation) is large right after the measurement target steps on the weight scale, such a period is excluded from the load value. It is preferable to use information representing the variation of load value used for calculating of the weight.

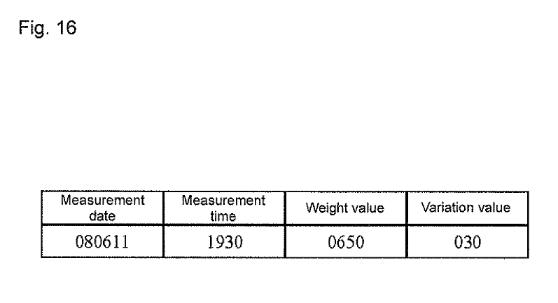

More specifically, as shown in FIG. 16, the measurement device 101 outputs data (output data) including a measurement date, a measurement time, a weight value, a variation value, and the like (FIG. 16 is an example of output data including a weight of 65.0 kg, a variation value (±) 30 g/sec, measured at 19:30 on Jun. 11, 2008). The variation value is a value representing the amount of fluctuation of the load value per second, and is a difference from, for example, an average value (an average value per second, a weight value, and the like).

Then, the evaluation unit 301 determines whether the biological information is reliable or not according to the variation value, and the display pattern instruction unit 302 causes the display unit 303 to display the biological information in a pattern according to the evaluation result of the evaluation unit 301. For example, when the fluctuation of load is equal to or more than ±20 g/sec, it is assumed that a human (user) is on the weight scale, and when the fluctuation of load is less than ±20 g/sec, it is assumed that a solid material is on the weight scale. In other words, when the fluctuation of load is equal to or more than ±20 g/sec, the biological information is evaluated as “reliable”, and when the fluctuation of load is less than ±20 g/sec, the biological information is evaluated as “unreliable”.

In the above description, the measurement device 101 calculates the validation value. Alternatively, the measurement device 101 may output the load value during measurement, and the administrator's terminal 104 may calculate the variation value from the load value.

### Sixth Embodiment

In the description of a sixth embodiment, the measurement device has a function of measuring a body composition, and a body composition is measured as biological information (in other words, a body composition monitor is used as the measurement device).

In the body composition monitor, there is a possibility of cheating, i.e., the user connects, instead of a user, a resistor having substantially the same impedance as a human to electrodes of the body composition monitor so as to cause the body composition monitor to calculate a false body composition value.

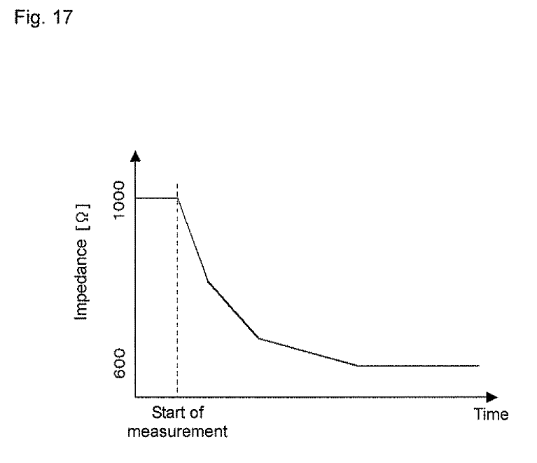

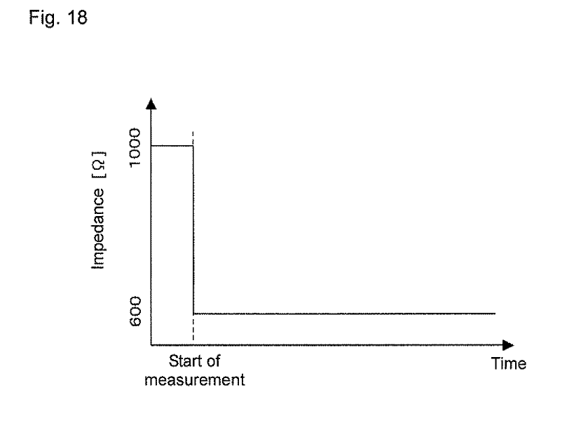

In general, when impedance of a human is measured by the body composition monitor, the impedance gradually decreases from the start of measurement, and eventually, settles to a substantially constant value, as shown in FIG. 17. This is because, right after the start of measurement, a contact between a human and electrodes is not sufficient, and when the humidity at the contact portion gradually increases, the state of contact is improved. On the other hand, when a resistor (electronic component) is connected to the electrodes, the impedance does not change as shown in FIG. 17 and is instantly stabilized as shown in FIG. 18. Therefore, whether the user correctly measures the body composition can be determined from the variation of impedance during measurement. Accordingly, in the present embodiment, in order to determine whether the body composition value is a false body composition value or not, information representing the variation of impedance during measurement is used as measurement state information. Whether the body composition of a human is measured or not can be determined if the variation of impedance can be obtained in a predetermined period from the start of measurement. Therefore, in the present embodiment, such a period is referred to as “during measurement”.

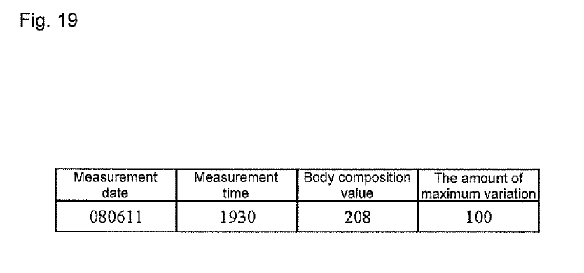

More specifically, as shown in FIG. 19, the measurement device 101 outputs data (output data) including a measurement date, a measurement time, a body composition value (body fat percentage), the amount of maximum variation, and the like (FIG. 19 is an example of output data including a body fat percentage of 20.8%, the amount of maximum variation of 100 Ω/sec, measured at 19:30 on Jun. 11, 2008). The amount of maximum variation is the maximum value of the amount of variation of body composition value (impedance) per second.

Then, the evaluation unit 301 evaluates whether the biological information is reliable or not according to the amount of maximum variation, and the display pattern instruction unit 302 causes the display unit 303 to display the biological information in a pattern according to the evaluation result of the evaluation unit 301. For example, when the amount of maximum variation is equal to or less than 300 Ω/sec, it is assumed that the body composition of a human (user) is measured, and when the amount of maximum variation is more than 300 Ω/sec, it is assumed that an object other than a human (such as resistor) is connected to the electrodes. In other words, when the amount of maximum variation is equal to or less than 300 Ω/sec, the biological information is evaluated as “reliable”, and when the amount of maximum variation is more than 300 Ω/sec, the biological information is evaluated as “unreliable”.

In the above description, the measurement device 101 calculates the amount of maximum variation. Alternatively, the measurement device 101 may output the value of impedance during measurement, and the administrator's terminal 104 may calculate the amount of maximum variation from the value of impedance.

Further, in the above description, the information representing the variation of impedance during measurement is used as the measurement state information. Alternatively, information representing the variation of body composition during measurement may be used as the measurement state information. Since the body composition is calculated from the impedance, even when information representing the variation of body composition during measurement is used, the biological information can be evaluated in the same manner as the above description.

### Seventh Embodiment

In the description of the seventh embodiment, the measurement device has a function of measuring a weight and a body composition, and a weight is measured as biological information (in other words, a weight scale having body composition measurement function is used).

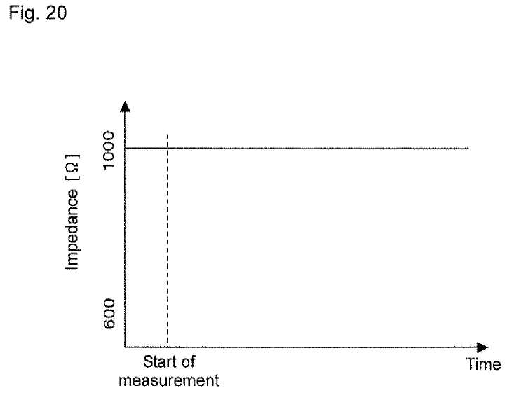

In general, when a solid material is on the weight scale, the solid material comes into contact (or nothing is in contact) with electrodes of the measurement device (electrodes for body composition measurement). Therefore, in such a case, when the solid material is a conducting material, the impedance measured by the body composition measurement function varies as shown in FIG. 18. When the solid material is an insulating material, the impedance remains substantially constant as shown in FIG. 20. Therefore, whether a human or a solid material is on the weight scale can be determined from the variation of impedance during measurement. Accordingly, in the present embodiment, in order to determine whether a human is on the weight scale or not, information representing the variation of impedance during measurement is used as measurement state information. Whether a human is on the weight scale or not can be determined if the variation of impedance can be obtained in a predetermined period from the start of measurement. Therefore, in the present embodiment, such a period is referred to as “during measurement”.

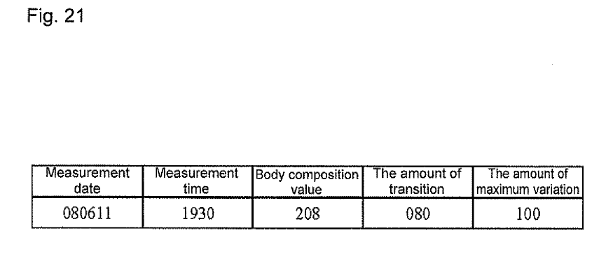

More specifically, as shown in FIG. 21, the measurement device 101 outputs data (output data) including a measurement date, a measurement time, a body composition value (body fat percentage), the amount of transition, the amount of maximum variation, and the like (FIG. 21 is an example of output data including a body fat percentage of 20.8%, the amount of transition of 80 Ω/sec, the amount of maximum variation of 100 Ω/sec, measured at 19:30 on Jun. 11, 2008). The amount of transition is a value representing the amount of variation (more specifically, an average value, the minimum value, and the like) of impedance per second.

Then, the evaluation unit 301 determines whether the biological information is reliable or not according to the amount of transition and the amount of maximum variation, and the display pattern instruction unit 302 causes the display unit 303 to display the biological information in a pattern according to the evaluation result of the evaluation unit 301. For example, when the amount of transition is equal to or more than 50 Ω/sec, it is assumed that a human is on the weight scale, and when the amount of transition less than 50 Ω/sec, it is assumed that a solid material is on the weight scale. On the other hand, when the amount of maximum variation is equal to or less than 300 Ω/sec, it is assumed that a human is on the weight scale, and when the amount of maximum variation is more than 300 Ω/sec, it is assumed that a solid material is on the weight scale. In other words, in a case where the amount of transition is equal to or more than 50 Ω/sec and the amount of maximum variation is equal to or less than 300 Ω/sec, the biological information is evaluated as “reliable”, and in a case other than the above, the biological information is evaluated as “unreliable”.

In the above description, the measurement device 101 calculates the amount of transition and the amount of maximum variation. Alternatively, the measurement device 101 may output the value of impedance during measurement, and the administrator's terminal 104 may calculate the amount of transition and the amount of maximum variation from the value of impedance.

Further, in the above description, the information representing the variation of impedance during measurement is used as the measurement state information. Alternatively, information representing the variation of body composition during measurement may be used as the measurement state information, in the same manner as the sixth embodiment.

By using the above-described measurement state information, the evaluation unit 301 can evaluate whether various kinds of biological information are reliable or not. For example, the evaluation unit 301 may be configured to switch the determination method according to the obtained biological information and the obtained measurement state information.

It should be noted that regardless of the types of measurement devices, a time needed to measure biological information with the measurement device is preferably used as the measurement state information.

When it takes a long time to measure the biological information, the user is likely to be unaccustomed to measurement, or the user is likely to try to do cheating. For example, when it takes too much time to measure a weight, the user may be adjusting the load by placing only one foot. When it takes too much time to measure a blood pressure and a body composition, the measurement method may be wrong, or the user may be repeating trial and error in order to do cheating (a measurement error is repeated).

When a time needed to measure biological information with the measurement device is used as measurement state information, such cheating can be detected. More specifically, the evaluation unit 301 may evaluate the biological information as “reliable” when the time needed for measurement is less than a predetermined time, and may evaluate the biological information as “unreliable” when the time needed for measurement is equal to or more than the predetermined time. It should be noted that the predetermined time may be different for each pieces of biological information.

The above-described criteria used for respective evaluation processings (criteria and threshold values for evaluation performed by the evaluation unit 301) are preferably changeable as necessary. The criteria for the determination performed by the evaluation unit 301 may be different for each person (for example, in many cases, a posture of a young person and a posture of an elderly person are different, and it is not appropriate to use the same criteria to make determination on such persons). For example, a doctor finds a health condition based on a result measured according to a measurement method appropriate for each individual person, and therefore, it would not be appropriate to use the same criteria for all the persons to evaluate biological information. Accordingly, by employing this structure, criteria appropriate for each person can be set, whereby biological information can be analyzed more accurately. For example, when biological information of a user is evaluated as “unreliable” many times for the same reason, the criteria are likely to be inappropriate. An administrator may appropriately reset criteria for such a user.

### <Modification>

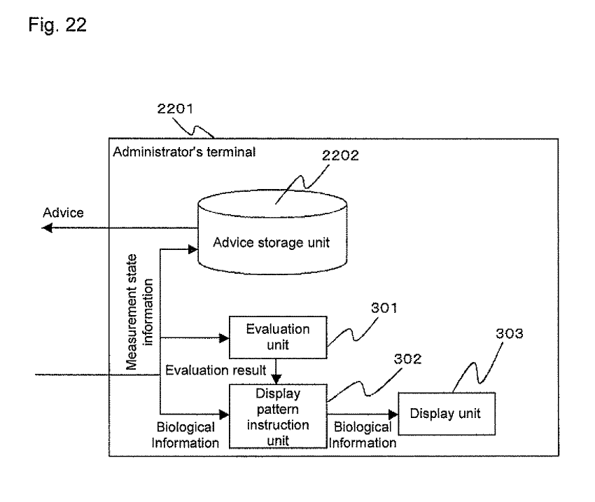

Subsequently, a modification of the biological information management system according to the present embodiment will be described. The biological information management system according to the present modification has the same structure as that of FIG. 1. However, as shown in FIG. 22, an administrator's terminal 2201 according to the present modification further includes an advice storage unit 2202 in addition to the structure of FIG. 3 (In FIG. 22, the same constituent elements as those described in FIG. 3 are denoted with the same reference numerals). In the below description, only portions different from the above structure will be described. The remaining portions are the same as the above structure, and the description thereabout is omitted.

The advice storage unit 2202 is a storage device for storing advices about measurement methods in association with measurement state information. The storage device may be a storage medium such as a non-volatile memory, a hard disk, and the like. In the present modification, after the administrator's terminal obtains (receives) measurement state information, the administrator's terminal outputs an advice corresponding to the received measurement state information from among the advices stored in the advice storage unit (corresponding to advice output means).

Specific examples of advices stored in the advice storage unit 2202 will be described. The stored advices of the measurement methods for a blood pressure monitor may include “measurement is performed with a correct posture”, “please raise your arm”, “please lower your arm”, and the like, according to the angle of the cuff unit. An advice such as “please perform measurement with a correct posture” may be included so as to prevent the user from knowing the criteria of determination. Such an advice may not be output on every measurement. For example, the advice may be outputted every time the measurement is performed five times, or the advice may be outputted when the evaluation unit 301 determines biological information as “unreliable” for five successive times. Further, the advice storage unit 2202 may store an advice for each type of measurement device, or may store an advice common to all measurement devices.

When the management device transmits an advice to a user according to a measurement state (information), the administrator of the management device needs to check the measurement state information, and transmit an advice. In the present modification, advices are automatically outputted according to measurement states as described above. Therefore, it is less cumbersome for the administrator (the administrator can save the trouble of transmitting such an advice). Further, the user is caused to understand that, when the user performs measurement according to a cheating measurement method, the user's measurement according to the cheating measurement method is known (to the administrator) and that the measurement method is a cheating. Therefore, cheating measurement can be reduced. On the other hand, the user is caused to understand that, when the user performs measurement according to a correct measurement method, the measurement method is correct. Therefore, the satisfaction of the user is considered to improve.

In addition to the above-mentioned advices, an advice such as “if you feel that the posture is uncomfortable during measurement, please take a comfortable posture” may be added. Therefore, the administrator can find out from subsequent measurement state information whether it is difficult to have the user perform measurement with a correct posture or not. When it is considered to be difficult to have the user perform measurement with a correct posture, the criteria of determination performed by the evaluation unit 301 may be changed as necessary based on measurement state information.

As described above, the biological information management system according to the present embodiment uses the simple method using the measurement state information to determine whether the biological information sent to the management device is reliable or not.

In the description of the present embodiment, the evaluation unit 301 evaluates the biological information as “reliable” or “unreliable”, for example. Alternatively, the evaluation unit 301 may calculate, based on the measurement state information, the degree of reliability representing how reliable the biological information is. More specifically, when a blood pressure is measured as the biological information, the degree of reliability may be calculated based on the amount of shift of a cuff angle, the number of body movements, and the like with respect to a predetermined value (for example, the degree of reliability is higher as the value is closer to the predetermined value, and the degree of reliability is lower as the amount of shift is larger.). When a weight is measured as the biological information, the degree of reliability may be calculated based on the amount of shift of a calibration load value, a variation value of load, the amount of transition of impedance, the amount of maximum variation of impedance, and the like with respect to a predetermined value. When a body composition is measured as the biological information, the degree of reliability may be calculated based on the amount of shift of the amount of maximum variation of impedance and the like with respect to a predetermined value. Further, the reliability may be calculated based on the amount of shift of the time needed for measurement with respect to a predetermined time. Therefore, the administrator can correctly analyze the biological information.

### DESCRIPTION OF SYMBOLS

100: Biological information management system
101: Measurement device
102: User's terminal
103: Data accumulation server
104: Administrator's terminal
201: Biological information measurement unit
202: Measurement state information generation unit
301: Evaluation unit
302: Display pattern instruction unit
303: Display unit
2201: Administrator's terminal
2202: Advice storage unit

1. A biological information management system comprising a measurement device for measuring biological information of a user and a management device for managing the biological information,
wherein the measurement device includes:
measurement state information generation means for generating measurement state information representing a state in which the biological information is measured when the biological information is measured; and
output means for outputting the biological information and the measurement state information generated when the biological information is measured, and
wherein the management device includes:
reception means for receiving the measurement state information and the biological information which are outputted by the output means; and
evaluation means for evaluating a reliability of the biological information received by the reception means based on the measurement state information received by the reception means.

2. The biological information management system according to claim 1, wherein the measurement device has a function of measuring a blood pressure,
and wherein when the blood pressure is measured as the biological information, the measurement state information is information representing at least any one of an angle of a cuff unit arranged on the measurement device for measuring the blood pressure, a variation of an acceleration of the cuff unit, and a variation of a pressure in the cuff unit during measurement.

3. The biological information management system according to claim 1, wherein the measurement device has a function of measuring a weight,
and wherein when the weight is measured as the biological information, the measurement state information is information representing a load value used for a zero-point calibration of the weight of the measurement device or a variation of a load during measurement.

4. The biological information management system according to claim 1, wherein the measurement device has a function of measuring a body composition,
and wherein when the body composition is measured as the biological information, the measurement state information is information representing a variation of the body composition or a variation of an impedance during measurement.

5. The biological information management system according to claim 1, wherein the measurement device has a function of measuring a weight and a body composition,
and wherein when the weight is measured as the biological information, the measurement state information is information representing a variation of the body composition or a variation of an impedance during measurement.

6. The biological information management system according to claim 1, wherein the measurement state information is a time it takes for the user to measure the biological information with the measurement device.

7. The biological information management system according to claim 1, wherein the management device includes:
storage means for storing advices about measurement methods in association with the measurement state information; and
advice output means for outputting an advice corresponding to the measurement state information received by the reception means from among the advices stored in the storage means.

8. The biological information management system according to claim 1, wherein the management device can change a criterion of evaluation performed by the evaluation means.

9. The biological information management system according to claim 1, wherein the management device includes display means causing a display unit of the management device to display the biological information in a pattern according to the evaluation result of the evaluation means.

10. The biological information management system according to claim 9, wherein the display means causes the display unit of the management device to display a graph of the biological information with a dot color, a dot shape, and/or a line type, according to the evaluation result of the evaluation means.

11. The biological information management system according to claim 9, wherein the display means causes the display unit of the management device to display only biological information in which the evaluation result of the evaluation means is a predetermined evaluation result.

12. A measurement device for measuring biological information of a user, the measurement device comprising:
measurement state information generation means for generating measurement state information representing a state in which the biological information is measured when the biological information is measured; and
output means for outputting the biological information and the measurement state information generated when the biological information is measured,
wherein the measurement state information is used by a management device managing the biological information to evaluate reliability of the biological information.
